(19) United States
(12) Patent Application Publication
(10) Pub. No.: US 2019/0382728 A1
(43) Pub. Date: Dec. 19, 2019

(54) Menstrual Blood Derived Angiogenesis Stimulatory Cells

(71) Applicants: Feng Lin, San Diego, CA (US); Fei Li, Beijing (CN); Dayong Zhang, Beijing (CN); Thomas Emanuel Ichim, San Dieog, CA (US)

(72) Inventors: Feng Lin, San Diego, CA (US); Fei Li, Beijing (CN); Dayong Zhang, Beijing (CN); Thomas Emanuel Ichim, San Dieog, CA (US)

(73) Assignee: Beijing Regenesis Biotechnology Co., Ltd, Beijing (CN)

(21) Appl. No.: 16/282,316

(22) Filed: Feb. 22, 2019

(51) Int. Cl. C12N 5/071

(52) U.S. Cl. CPC C12N 5/0682; C12N 2502/115

(57) ABSTRACT

Disclosed are angiogenesis stimulatory mesenchymal-like cells derived from menstrual blood, originating from endometrium and expressing increased levels of the protein CD39 as compared to other cells found from the menstrual blood. In one embodiment, menstrual blood mononuclear cells are plated on adherent culture and allowed to grow in a manner permitting expansion of mesenchymal-like cells. Said expanded cells are selected for expression of CD39 and utilized for stimulation of angiogenesis. In other embodiments, menstrual blood derived adherent cells are cultured with platelet derived factors in order to augment angiogenic ability, directly, or through upregulation of CD39 expression.

### BACKGROUND OF THE INVENTION

### Field of the Invention

The invention pertains to the area of cellular therapies, more specifically, the invention pertains to the area of cellular therapies derived from naturally regenerating cells, more specifically, the invention pertains to cellular therapies derived from the endometrium.

### Description of the Related Art

Cellular therapies for treatment of degenerative disorders are an attractive therapeutic option. In particular, studies have shown that cells of mesenchymal origin possess various attributes making them desirable for use in conditions where angiogenesis is desirable.

Angiogenesis therapy has been described as a “biological bypass”, the idea being that through administration of agents capable of inducing collateralization, a more natural type of “bypass” can be achieved. Indeed it has been observed that ischemic muscles secrete angiogenic factors in response to hypoxia and that to some extent natural angiogenesis does occur in animal models of critical limb ischemia (CLI) and in humans [1, 2]. One of the angiogenic factors noted in many ischemic conditions, including cardiac ischemia, stroke, and CLI is vascular endothelial growth factor (VEGF) [3-5]. In 1994, Isner's group sought to enhance ischemia-associated angiogenesis using single bolus intra-arterial administration of VEGF-165 in a rabbit model of CLI. Rabbits with resected femoral arteries demonstrated augmentation of perfusion, increased capillary density, and overall better function as compared to control rabbits [6]. Subsequent experiments sought to optimize therapeutic effect using different dosing schedules. Daily VEGF-165 administration for 10 days subsequent to ligation and resection of the external iliac artery and femoral artery, respectively was performed [7]. Not only was dose-dependent increase in collateralization observed, but rabbits receiving the highest dose of VEGF-165 (1 mg) had no incidence of calf muscle atrophy and distal limb necrosis, whereas this was present in 85.7% of control rabbits. A similar study in the rabbit model of CLI demonstrated superior benefit in terms of limb function in animals receiving VEGF by osmotic pump for 28-days in comparison to nitroglycerin or saline treatment [8]. A variety of studies have been performed with VEGF protein which confirmed the proangiogenic, as well as angiogenesis-independent muscle reparative properties in the limb ischemia model [9-11]. These positive preclinical data unfortunately were not successfully reproduced in the clinic. Trials using VEGF protein [12], or DNA, did not show significant benefit at reducing leg amputations in a double blind setting [11, 13]. One of the concerns with VEGF therapy is its vasodilatory effects, as well as induction of blood vessels that are relatively immature and “leaky”.

Another approach involved use of the cytokine fibroblast growth factor-1 (FGF-1). Given that FGF-1 is considered “upstream” of VEGF, it is believed to stimulate numerous angiogenic processes so as to result in creation of more mature vessels [14]. Indeed, it is believed that one of the differences between tumor neo-vasculature, which is characteristically leaky, and neo-vasculature associated with physiological angiogenesis, is the ratio of VEGF to FGF [15]. Specifically, it was demonstrated both in animal models [16] and in clinical trials [17] that muscular ischemia is associated with up-regulation of FGF gene transcription, suggesting that this is part of the endogenous pro-angiogenic response to ischemia. The critical role of FGF in endogenous angiogenesis was conclusively demonstrated in FGF knockout mice, which displayed inhibited ability to heal post-wounding [18]. Although FGF-1 gene therapy has clinically been used in CLI patients with some improvement in ABI and perfusion, statistically powered randomized clinical trials have not been conducted to date [19].

It is known that additive and/or synergistic effects are observed in terms of neoangiogenesis when several angiogenic factors are combined. For example, Cao et al demonstrated synergy between administration of PDGF-BB and FGF-2 in terms of increasing blood vessel formation and function in the femoral artery ligation model in rats and rabbits [20]. Similarly, in cancer angiogenesis, it is known that several tumor-derived angiogenic factors synergize for acceleration of neovascularization [21]. Accordingly, investigators have attempted to activate upstream mediators of several angiogenic signals through transfection of genes encoding transcription factors such as HIF-1 alpha [22]. In fact, this approach has been demonstrated to be superior to VEGF gene administration in terms of new capillary sprouting. In a Phase I dose-escalating trial, transfection of HIF-1 alpha into CLI patients demonstrated tolerability with some indication of efficacy [23].

In conclusion, while administration of angiogenic factors to patients with CLI does induce some benefit in early trials, data from randomized trials to date do not support widespread use. The transfection of upstream transcription factors such as HIF-1 alpha is a promising approach since it mimics natural angiogenesis in that a plurality of growth factors are induced following transfection [22, 24]. However clinical results are too premature to draw firm conclusions. Additionally, the transfection of foreign genes may possess unintended consequences in the long run due to the uncontrolled nature of the transfected gene insert. The current invention aims to overcome limitations in the use of angiogenesis therapy by utilizing natural abilities of cells derived from the endometrium to support angiogenesis, and subsequently, when placental development occurs, to support immune evasion.

### BRIEF DESCRIPTION OF THE DRAWINGS

FIG. 1. Comparison of VEGF production in CD39+ and CD39− ERCs and enhancement of VEGF production in ERCs by PRP. After obtaining ERCs, the cells were separated into CD39+ and CD39− subset using magnetic activated cellular sorting (MACS). Unseparated state ERC was labeled as ERC non-fractionated. Cells were seeded in 96-well plates, each well 20000 cells. Completed DMEM media was used, and PRR was added at 0, 2%, 5% and 10%, respectively. After 24 hours culture, supernatant was extracted and analyzed for cytokine production by ELISA using VEGF kit (R&D system).

FIG. 2. Comparison of HGF production by CD39+ and CD39− ERCs and enhancement of HGF production in ERCs by PRP. After obtaining ERCs, the cells were separated into CD39+ and CD39− subset using magnetic activated cellular sorting (MACS). Unseparated state ERC was labeled as ERC non-fractionated. Cells were seeded in 96-well plates, each well 20000 cells. Completed DMEM media was used, and platelet rich plasma was added at 2%, 5% and 10%. After 24 hours culture, supernatant was extracted and analyzed for cytokine production by ELISA using HGF quantitative kit (R&D system).

FIG. 3. Comparison of PDGF-BB production by CD39+ and CD39− ERCs and enhancement of PDGF-BB production in ERCs by PRP. After obtaining ERCs, the cells were separated into CD39+ and CD39− subset using magnetic activated cellular sorting (MACS). Unseparated state ERC was labeled as ERC non-fractionated. Cells were seeded in 96-well plates, each well 20000 cells. Completed DMEM media was used, and platelet rich plasma was added at 2%, 5% and 10%. After 24 hours culture, supernatant was extracted and analyzed for cytokine production by ELISA using PDGF-BB quantitative kit (R&D system).

FIG. 4. Stimulation of ERC angiogenesis by CD39 expression and enhancement of angiogenesis by PRP. After obtaining ERCs, the cells were separated into CD39+ and CD39− subset using magnetic activated cellular sorting (MACS). Unseparated state ERC was labeled as ERC non-fractionated. Cells were seeded in 96-well plates, each well 20000 cells. Completed DMEM media was used for culture. After 24 hours culture, supernatant was extracted and harvested. In order to replicate angiogenic processes in vitro, a culture of human umbilical vein endothelial cells (HUVEC) is performed in 96-well plates. HUVEC cells were plated at a concentration of 20000 cells per well. Cells were subsequently incubated at the indicated ratio with HUVEC cells in a total of 200 ul of media per well for 72 hours. 1 uCurie/ml of tritiated thymidine was added in the last 12 hours of culture and proliferation was quantified scintillation counting. Proliferation is expressed as counts per minute.

### DETAILED DESCRIPTION OF THE INVENTION

The invention teaches isolation of a potent angiogenic type of endometrially derived cell expressing the marker CD39. The angiogenic potency of endometrial cells can be increased by isolation of endometrial stem cells selected for expression of CD39, or alternatively can be augmented by treatment with platelet rich plasma. In the practice of the invention, platelet rich plasma may be used to augment angiogenic activity of endometrial derived cells by increasing the number of cells expressing CD39 in tissue culture. In other embodiments of the invention, cells already expressing CD39 are endowed with augmentation of angiogenic activity by treatment with platelet rich plasma.

In one specific embodiment, endometrial stem cells, or otherwise defined as “endometrial regenerative cells” are derived from menstrual blood. Menstrual blood is collected from a healthy female subject after menstrual blood flow initiated. Collection is performed in a urine cup and then transferred into a 5 ml tube with 0.2 ml amphotericin B (Sigma-Aldrich, St Louis, Mo.), 0.2 ml penicillin/streptomycin (Sigma 50 ug/nl) and 0.1 ml EDTA-Na2 (Sigma) in phosphate buffered saline (PBS). Mononuclear cells derived from menstrual blood were separated by Ficoll-Paque (Fisher Scientific, Portsmouth N.H.) according to the instruction and washed in PBS. Cells are subsequently cultured in a Petri dish (Corning, Acton, Mass.) containing DMEM medium supplemented with 1% penicillin/streptomycin, 1% amphotericin B, 1% glutamine and 20% FBS (completed DMEM). Media is changed the next day. Adherent cells are detached by trypsin and cultured in a T75 flask (Fisher Scientific, Portsmouth N.H.) at 1×10⁵ cells. The cells were then subcultured and passaged twice a week. Cloning of cells was accomplished by plating cells at a concentration of approximately 1 cell per well in 96 well plates (Corning, Acton, Mass.). Subsequent to growth of the cells, the CD39 expressing fraction is separated and subsequently grown. Separation may be performed by numerous means, for example, utilizing magnetic activated cell sorting (MACS). Other means involve flow cytometry sorting, cellular panning, or other means involving selective purification of cells expressing higher levels of CD39.

In another embodiment, expression of CD39 is upregulated by treatment with platelet rich plasma. It is known that numerous growth factors, cytokines and peptides are released from activated platelets, one strategy to therapeutically leverage this fact is to prepare an autologous platelet concentrate suspended in plasma, also known as platelet-rich plasma (PRP). In one embodiment of the invention, PRP is utilized to augment expression ofSeveral means of preparing PRP are known in the art, some of which are described in the following and incorporated by reference [25, 26]. Examples of devices used for generation of PRP include SmartPReP, 3iPCCS, Sequestra, Secquire, CATS, Interpore Cross, Biomet GPS, and Harvest's BMAC [27]. Other means of generating PRP are described in U.S. Pat. Nos. 5,585,007, 5,599,558, 5,614,204, 6,214,338; 6,010,627; 5,165,928; 6,303,112; 6,649,072, 6,649,072, which are incorporated by reference. These devices may be utilized to generate PRP for treatment of endometrial stem cells for augmentation of CD39 expressing cells, as well as increasing potency of endometrial stem cells for stimulation of angiogenesis.

In various embodiments of the present invention, the platelet plasma composition may be obtained by sequestering platelets from whole blood or bone marrow through centrifugation, for example into three strata: (1) platelet rich plasma; (2) platelet poor plasma; and (3) fibrinogen. When using platelets from one of the strata, e.g., the PRP from blood, one may use the platelets whole or their contents may be extracted and concentrated into a platelet lysate through a cell membrane lysis procedure using thrombin and/or calcium chloride, for example. When choosing whether to use the platelets whole or as a lysate, one may consider the rate at which one desires regeneration and/or tissue healing (which may include the formation of scar tissue without regeneration or healing of a herniated or torn disc). In some embodiments the lysate will act more rapidly than the PRP (or platelet poor plasma from bone marrow). Notably, platelet poor plasma that is derived from bone marrow has a greater platelet concentration than platelet rich plasma from blood, also known as platelet poor/rich plasma, (“PP/RP” or “PPP”). PP/RP or PPP may be used to refer to platelet poor plasma derived from bone marrow, and in some embodiments, preferably PP/RP is used or PRP is used as part of the composition for disc regeneration. (By convention, the abbreviation PRP refers only to compositions derived from peripheral blood and PPP (or PP/RP) refers to compositions derived from bone marrow.)

The endometrial stem cells utilized in the invention are generated, in one embodiment, by outgrowth from a biopsy of the donor endometrium. In some embodiments endometrial cells are used from young donors. In another embodiment endometrial stem cells are transfected with genes to allow for enhanced growth and overcoming of the Hayflick limit. Subsequent to derivation of cells expansion in culture using standard cell culture techniques. In one embodiment, the starting material is menstrual blood. The samples are shipped in a 2-8.degree. C. refrigerated shipper back to the manufacturing facility. In one embodiment, after arrival at the manufacturing facility, the biopsy is inspected and, upon acceptance, transferred directly to the manufacturing area. Upon initiation of the process, the biopsy tissue is then washed prior to enzymatic digestion. After washing, a Liberase Digestive Enzyme Solution is added without mincing, and the biopsy tissue is incubated at 37.0.+−0.2.degree. C. for one hour. Time of biopsy tissue digestion is a critical process parameter that can affect the viability and growth rate of cells in culture. Liberase is a collagenase/neutral protease enzyme cocktail obtained formulated from Lonza Walkersville, Inc. (Walkersville, Md.) and unformulated from Roche Diagnostics Corp. (Indianapolis, Ind.). Alternatively, other commercially available collagenases may be used, such as Serva Collagenase NB6 (Helidelburg, Germany). After digestion, Initiation Growth Media (IMDM, GA, 10% Fetal Bovine Serum (FBS)) is added to neutralize the enzyme, cells are pelleted by centrifugation and resuspended in 5.0 mL Initiation Growth Media. Alternatively, centrifugation is not performed, with full inactivation of the enzyme occurring by the addition of Initiation Growth Media only. Initiation Growth Media is added prior to seeding of the cell suspension into a T-175 cell culture flask for initiation of cell growth and expansion. A T-75, T-150, T-185 or T-225 flask can be used in place of the T-75 flask. Cells are incubated at 37.+−0.2.0.degree. C. with 5.0.+−0.1.0% CO.sub.2 and fed with fresh Complete Growth Media every three to five days. All feeds in the process are performed by removing half of the Complete Growth Media and replacing the same volume with fresh media. Alternatively, full feeds can be performed. Cells should not remain in the T-175 flask greater than 30 days prior to passaging. Confluence is monitored throughout the process to ensure adequate seeding densities during culture splitting. When cell confluence is greater than or equal to 40% in the T-175 flask, they are passaged by removing the spent media, washing the cells, and treating with Trypsin-EDTA to release adherent cells in the flask into the solution. Cells are then trypsinized and seeded into a T-500 flask for continued cell expansion. Alternately, one or two T-300 flasks, One Layer Cell Stack (1 CS), One Layer Cell Factory (1 CF) or a Two Layer Cell Stack (2 CS) can be used in place of the T-500 Flask. Morphology is evaluated at each passage and prior to harvest to monitor the culture purity throughout the culture purity throughout the process. Morphology is evaluated by comparing the observed sample with visual standards for morphology examination of cell cultures. The cells display typical fibroblast morphologies when growing in cultured monolayers. Cells may display either an elongated, fusiform or spindle appearance with slender extensions, or appear as larger, flattened stellate cells which may have cytoplasmic leading edges. A mixture of these morphologies may also be observed. Fibroblasts in less confluent areas can be similarly shaped, but randomly oriented. The presence of keratinocytes in cell cultures is also evaluated. Endometrial stem cells appear round and irregularly shaped and, at higher confluence, they appear organized in a cobblestone formation. At lower confluence, keratinocytes are observable in small colonies. Cells are incubated at 37.+−0.2.0.degree. C. with 5.0.+−0.1.0% CO2 and passaged every three to five days in the T-500 flask and every five to seven days in the ten layer cell stack (10CS). Cells should not remain in the T-500 flask for more than 10 days prior to passaging. Quality Control (QC) release testing for safety of the Bulk Drug Substance includes sterility and endotoxin testing. When cell confluence in the T-500 flask is .gtoreq.95%, cells are passaged to a 10 CS culture vessel. Alternately, two Five Layer Cell Stacks (5 CS) or a 10 Layer Cell Factory (10 CF) can be used in place of the 10 CS. 10CS. Passage to the 10 CS is performed by removing the spent media, washing the cells, and treating with Trypsin-EDTA to release adherent cells in the flask into the solution. Cells are then transferred to the 10 CS. Additional Complete Growth Media is added to neutralize the trypsin and the cells from the T-500 flask are pipetted into a 2 L bottle containing fresh Complete Growth Media. The contents of the 2 L bottle are transferred into the 10 CS and seeded across all layers. Cells are then incubated at 37.+−0.2.0.degree. C. with 5.0.+−0.1.0% CO.sub.2 and fed with fresh Complete Growth Media every five to seven days. Cells should not remain in the 10CS for more than 20 days prior to passaging. In one embodiment, the passaged dermal fibroblasts are rendered substantially free of immunogenic proteins present in the culture medium by incubating the expanded fibroblasts for a period of time in protein free medium, Primary Harvest When cell confluence in the 10 CS is 95% or more, cells are harvested. Harvesting is performed by removing the spent media, washing the cells, treating with Trypsin-EDTA to release adherent cells into the solution, and adding additional Complete Growth Media to neutralize the trypsin. Cells are collected by centrifugation, resuspended, and in-process QC testing performed to determine total viable cell count and cell viability.

In some embodiments, when large numbers of cells are required after receiving cell count results from the primary 10 CS harvest, an additional passage into multiple cell stacks (up to four 10 CS) is performed. For additional passaging, cells from the primary harvest are added to a 2 L media bottle containing fresh Complete Growth Media. Resuspended cells are added to multiple cell stacks and incubated at 37.+−0.2.0.degree. C. with 5.0.+−0.1.0% CO.sub.2. The cell stacks are fed and harvested as described above, except cell confluence must be 80% or higher prior to cell harvest. The harvest procedure is the same as described for the primary harvest above. A mycoplasma sample from cells and spent media is collected, and cell count and viability performed as described for the primary harvest above. The method decreases or eliminates immunogenic proteins be avoiding their introduction from animal-sourced reagents. To reduce process residuals, cells are cryopreserved in protein-free freeze media, then thawed and washed prior to prepping the final injection to further reduce remaining residuals. If additional Drug Substance is needed after the harvest and cryopreservation of cells from additional passaging is complete, aliquots of frozen Drug Substance—Cryovial are thawed and used to seed 5 CS or 10 CS culture vessels. Alternatively, a four layer cell factory (4 CF), two 4 CF, or two 5 CS can be used in place of a 5 CS or 10 CS. A frozen cryovial(s) of cells is thawed, washed, added to a 2 L media bottle containing fresh Complete Growth Media and cultured, harvested and cryopreserved as described above. The cell suspension is added Cell confluence must be 80% or more prior to cell harvest.

At the completion of culture expansion, the cells are harvested and washed, then formulated to contain 1.0-2.7.times.10.sup.7 cells/mL, with a target of 2.2.times.10.sup.7 cells/mL. Cells may be treated with PRP subsequent to expansion, or more preferably, during the expansion process. Alternatively CD39 cells may be selected previous to expansion.

Alternatively, the target can be adjusted within the formulation range to accommodate different indication doses. The drug substance consists of a population of viable, endometrial stem cells suspended in a cryopreservation medium consisting of Iscove's Modified Dulbecco's Medium (IMDM) and Profreeze-CDM™ (Lonza, Walkerville, Md.) plus 7.5% dimethyl sulfoxide (DMSO). Alternatively, a lower DMSO concentration may be used in place of 7.5% or CryoStor™ CS5 or CryoStor™ CS10 (BioLife Solutions, Bothell, Wash.) may be used in place of IMDM/Profreeze/DMSO. In addition to cell count and viability, purity/identity of the Drug Substance is performed and must confirm the suspension contains 98% or more endometrial stem cells. The purity/identify assay employs fluorescent-tagged antibodies against CD90 and CD 104 (cell surface markers for fibroblast and keratinocyte cells, respectively) to quantify the percent purity of a fibroblast cell population. CD90 (Thy-1) is a 35 kDa cell-surface glycoprotein. Antibodies against CD90 protein have been shown to exhibit high specificity to human fibroblast cells. CD104, integrin .beta.4 chain, is a 205 kDa transmembrane glycoprotein which associates with integrin .alpha.6 chain (CD49f) to form the .alpha.6/.beta.4 complex.

In another embodiment, cells can be processed on poly blend 2D microcarriers such as BioNOC II® and FibraCel® using an automatic bellow system, such as FibraStage™ (New Brunswick Scientific, Edison, N.J.) or BelloCell® (Cesco Bioengineering, distributed by Bellco Biotechnology, Vineland, N.J.) in place of the spinner flask apparatus. Cells from the T-175 (or alternatives) or T-500 flask (or alternatives) are passaged into a bellow bottle containing microcarriers with the appropriate amount of Complete Growth Media, and placed into the system. The system pumps media over the microcarriers to feed cells, and draws away media to allow for oxygenation in a repeating fixed cycle. Cells are monitored, fed, washed and harvested in the same sequence as described above. Alternatively, cells can be processed using automated systems. After digestion of the biopsy tissue or after the first passage is complete (T-175 flask or alternative), cells may be seeded into an automated device. One method is an Automated Cellular Expansion (ACE) system, which is a series of commercially available or custom fabricated components linked together to form a cell growth platform in which cells can be expanded without human intervention. Cells are expanded in a cell tower, consisting of a stack of disks capable of supporting anchorage-dependent cell attachment. The system automatically circulates media and performs trypsinization for harvest upon completion of the cell expansion stage.

For use of the endometrial stem cells, the PRP composition may comprise a PRP derived from a human or animal source of whole blood. The PRP may be prepared from an autologous source, an allogenic source, a single source, or a pooled source of platelets and/or plasma. To derive the PRP, whole blood may be collected, for example, using a blood collection syringe. The amount of blood collected may depend on a number of factors, including, for example, the amount of PRP desired, the health of the patient, the severity or type of the arrhythmia, the availability of prepared PRP, or any suitable combination of factors. Any suitable amount of blood may be collected. For example, about 20 cc to about 150 cc of blood may be drawn. More specifically, about 27 cc to about 110 cc or about 27 cc to about 55 cc of blood may be withdrawn. In some embodiments, the blood may be collected from a patient who may be presently suffering, or who has previously suffered from, a cardiac arrhythmia. PRP made from a patient's own blood may significantly reduce the risk of adverse reactions or infection. The PRP may be prepared in any suitable way. For example, the PRP may be prepared from whole blood using a centrifuge. The whole blood may or may not be cooled after being collected. Isolation of platelets from whole blood depends upon the density difference between platelets and red blood cells. The platelets and white blood cells are concentrated in the layer (i.e., the “buffy coat”) between the platelet depleted plasma (top layer) and red blood cells (bottom layer). For example, a bottom buoy and a top buoy may be used to trap the platelet-rich layer between the upper and lower phase. This platelet-rich layer may then be withdrawn using a syringe or pipette. Generally, at least 60% or at least 80% of the available platelets within the blood sample can be captured. These platelets may be resuspended in a volume that may be about 3% to about 20% or about 5% to about 10% of the sample volume. In an exemplary embodiment, about 55 cc of blood may be withdrawn into a 60 cc syringe (or another suitable syringe) that contains about 5 cc of an anticoagulant, such as a citrate dextrose solution. The syringe may be attached to an apheresis needle, and primed with the anticoagulant. Blood (about 27 cc to about 55 cc) may be drawn from the patient using standard aseptic practice. In some embodiments, a local anesthetic such as anbesol, benzocaine, lidocaine, procaine, bupivicaine, or any appropriate anesthetic known in the art may be used to anesthetize the insertion area. In some examples, the blood may then be centrifuged using a gravitational platelet system, such as the Cell Factor Technologies GPS System® centrifuge. The blood-filled syringe containing between about 20 cc to about 150 cc of blood (e.g., about 55 cc of blood) and about 5 cc citrate dextrose may be slowly transferred to a disposable separation tube which may be loaded into a port on the GPS centrifuge. The sample may be capped and placed into the centrifuge. The centrifuge may be counterbalanced with about 60 cc sterile saline, placed into the opposite side of the centrifuge. Alternatively, if two samples are prepared, two GPS disposable tubes may be filled with equal amounts of blood and citrate dextrose. The samples may then be spun to separate platelets from blood and plasma. The samples may be spun at about 2000 rpm to about 5000 rpm for about 5 minutes to about 30 minutes. For example, centrifugation may be performed at 3200 rpm for extraction from a side of the separation tube and then isolated platelets may be suspended in about 3 cc to about 5 cc of plasma by agitation. The PRP may then be extracted from a side port using, for example, a 10 cc syringe. If about 55 cc of blood may be collected from a patient, about 5 cc of PRP may be obtained. The PRP composition may be delivered to help facilitate perispinal angiogenesis. In one embodiment of the invention PRP is administered via a 27 gauge needle, into the lumbar muscles in proximity to the area of occlusion identified by MR aortography. The injection site is limited to an area within a 1 cm diameter around the area of occlusion. The needle is inserted at a 45° angle directly into the back at a depth no greater than 2 cm. The axis of the needle is inserted toward the periphery of the vascular bed.

In some embodiments of the invention, growth factors are added to the PRP mixture to enhance angiogenesis, examples of growth factors are listed has follows and papers describing their use are incorporated by reference: TPO [28-32], SCF [33-35], IL-1 [36-38], IL-3 [39-41], IL-6 [42-45], IL-7 [46-49], IL-11 [37, 50-52], flt-3L [53-56], G-CSF [57-59], GM-CSF [42, 60], Epo [61-63], FGF-1 [64, 65], FGF-2 [66, 67], FGF-4 [68, 69], FGF-20 [70], IGF [71-74], EGF [75, 76], NGF [77, 78], LIF [79, 80], PDGF [81-83], BMPs [84-88], activin-A [89], VEGF [90], forskolin [91, 92], and glucocorticoids [93].

### EXAMPLES

### Example 1

### Superior Production of VEGF by CD39 Menstrual Blood Derived Cells and Enhancement by PRP

Menstrual blood was collected from a healthy female subject after menstrual blood flow initiated. Collection was performed in a urine cup and then transferred into a 5 ml tube with 0.2 ml amphotericin B (Sigma-Aldrich, St Louis, Mo.), 0.2 ml penicillin/streptomycin (Sigma 50 ug/nl) and 0.1 ml EDTA-Na2 (Sigma) in phosphate buffered saline (PBS). Mononuclear cells derived from menstrual blood were separated by Ficoll-Paque (Fisher Scientific, Portsmouth N.H.) according to the instruction and washed in PBS. Cells were subsequently cultured in a Petri dish (Corning, Acton, Mass.) containing DMEM medium supplemented with 1% penicillin/streptomycin, 1% amphotericin B, 1% glutamine and 20% FBS (completed DMEM). Media was changed the next day. Adherent cells were detached by trypsin and cultured in a T75 flask (Fisher Scientific, Portsmouth N.H.) at 1×10⁵ cells. The cells were then subcultured and passaged twice a week. PRP is added at the indicated concentration.

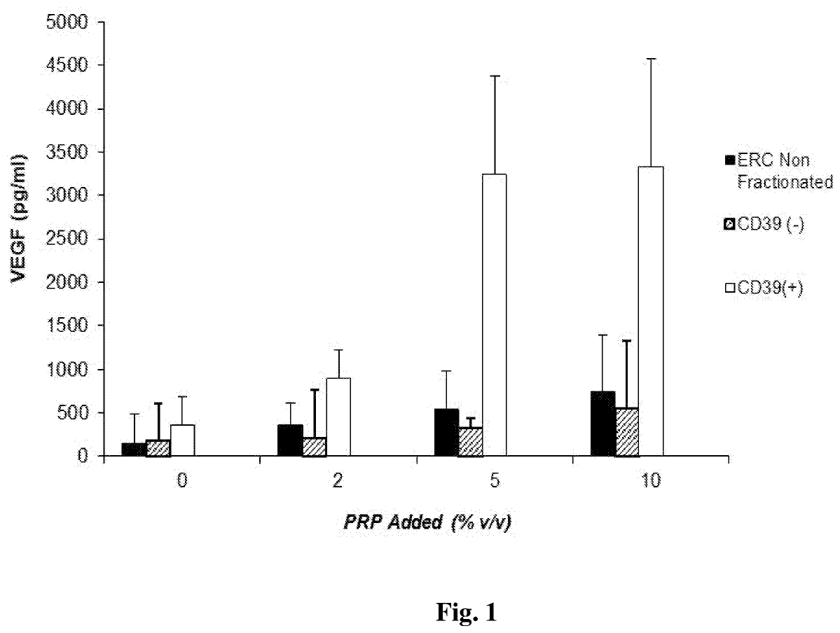

Cells were cultured in an unseparated state (ERC non-fractionated), into CD39+ and CD39− subsets using magnetic activated cellular sorting (MACS). Cells were plated with indicated concentrations of platelet rich plasma and cultured for 24 hours in DMEM media. Supernatant was extracted and analyzed for cytokine production by ELISA. The production of VEGF in non-fractionated ERCs, CD39+ ERCs and CD39− ERCs with and without treatment of PRP is shown in FIG. 1.

### Example 2

### Superior Production of HGF by CD39 Menstrual Blood Derived Cells and Enhancement by PRP

Menstrual blood was collected from a healthy female subject after menstrual blood flow initiated. Collection was performed in a urine cup and then transferred into a 5 ml tube with 0.2 ml amphotericin B (Sigma-Aldrich, St Louis, Mo.), 0.2 ml penicillin/streptomycin (Sigma 50 ug/nl) and 0.1 ml EDTA-Na2 (Sigma) in phosphate buffered saline (PBS). Mononuclear cells derived from menstrual blood were separated by Ficoll-Paque (Fisher Scientific, Portsmouth N.H.) according to the instruction and washed in PBS. Cells were subsequently cultured in a Petri dish (Corning, Acton, Mass.) containing DMEM medium supplemented with 1% penicillin/streptomycin, 1% amphotericin B, 1% glutamine and 20% FBS (completed DMEM). Media was changed the next day. Adherent cells were detached by trypsin and cultured in a T75 flask (Fisher Scientific, Portsmouth N.H.) at 1×10⁵ cells. The cells were then subcultured and passaged twice a week. PRP was added at the indicated concentration.

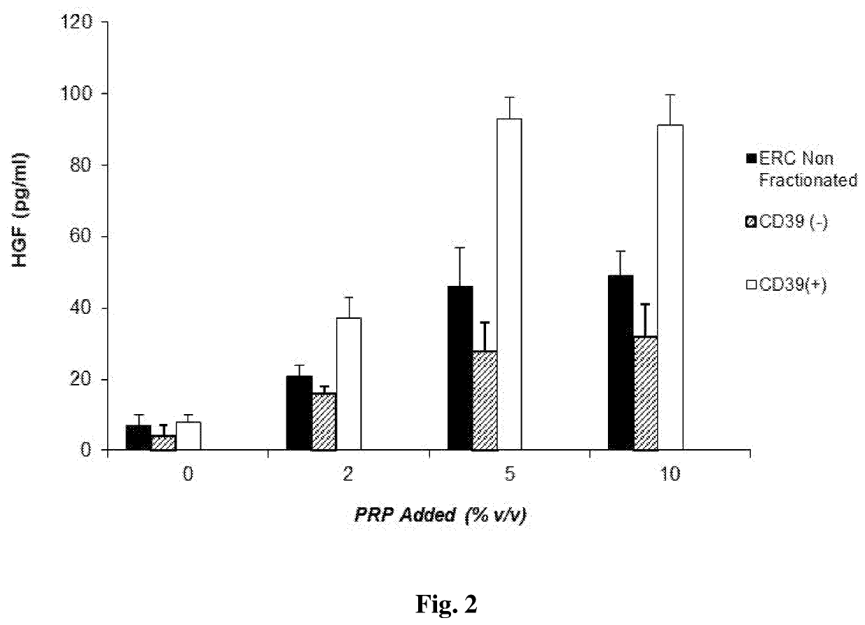

Cells were cultured in an unseparated state (ERC non-fractionated), into CD39+ and CD39− subsets using magnetic activated cellular sorting (MACS). Cells were plated with indicated concentrations of platelet rich plasma and cultured for 24 hours in DMEM media. Supernatant was extracted and analyzed for cytokine production by ELISA. The production of HGF in non-fractionated ERCs, CD39+ ERCs and CD39− ERCs with and without treatment of PRP is shown in FIG. 2.

### Example 3

### Superior Production of PDGF-BB by CD39 Menstrual Blood Derived Cells and Enhancement by PRP

Menstrual blood was collected from a healthy female subject after menstrual blood flow initiated. Collection was performed in a urine cup and then transferred into a 5 ml tube with 0.2 ml amphotericin B (Sigma-Aldrich, St Louis, Mo.), 0.2 ml penicillin/streptomycin (Sigma 50 ug/nl) and 0.1 ml EDTA-Na2 (Sigma) in phosphate buffered saline (PBS). Mononuclear cells derived from menstrual blood were separated by Ficoll-Paque (Fisher Scientific, Portsmouth NH) according to the instruction and washed in PBS. Cells were subsequently cultured in a Petri dish (Corning, Acton, Mass.) containing DMEM medium supplemented with 1% penicillin/streptomycin, 1% amphotericin B, 1% glutamine and 20% FBS (completed DMEM). Media was changed the next day. Adherent cells were detached by trypsin and cultured in a T75 flask (Fisher Scientific, Portsmouth NH) at 1×10⁵ cells. The cells were then subcultured and passaged twice a week. PRP was added at the indicated concentration.

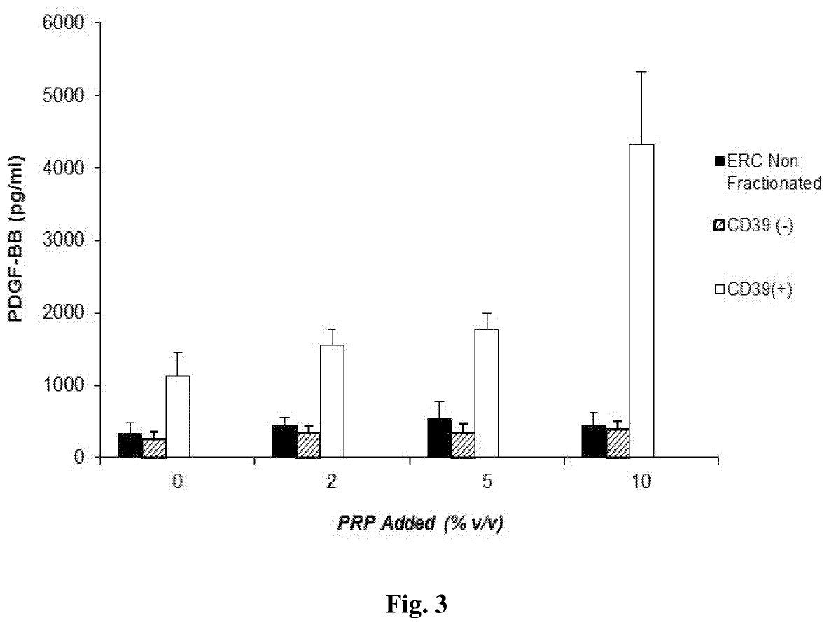

Cells were cultured in an unseparated state (ERC non-fractionated), into CD39+ and CD39− subsets using magnetic activated cellular sorting (MACS). Cells were plated with indicated concentrations of platelet rich plasma and cultured for 24 hours in DMEM media. Supernatant was extracted and analyzed for cytokine production by ELISA. The production of PDGF-BB in non-fractionated ERCs, CD39+ ERCs and CD39− ERCs with and without treatment of PRP is shown in FIG. 3.

### Example 4

### Superior Stimulation of Angiogenesis by CD39 Expressing Menstrual Derived Stem Cells and Enhancement by PRP

Menstrual blood was collected from a healthy female subject after menstrual blood flow initiated. Collection was performed in a urine cup and then transferred into a 5 ml tube with 0.2 ml amphotericin B (Sigma-Aldrich, St Louis, Mo.), 0.2 ml penicillin/streptomycin (Sigma 50 ug/nl) and 0.1 ml EDTA-Na2 (Sigma) in phosphate buffered saline (PBS). Mononuclear cells derived from menstrual blood were separated by Ficoll-Paque (Fisher Scientific, Portsmouth N.H.) according to the instruction and washed in PBS. Cells were subsequently cultured in a Petri dish (Corning, Acton, Mass.) containing DMEM medium supplemented with 1% penicillin/streptomycin, 1% amphotericin B, 1% glutamine and 20% FBS (completed DMEM). Media was changed the next day. Adherent cells were detached by trypsin and cultured in a T75 flask (Fisher Scientific, Portsmouth N.H.) at 1×10⁵ cells. The cells were then subcultured and passaged twice a week. Platelet rich plasma was added at the indicated concentration.

Cells were cultured in an unseparated state (ERC non-fractionated), into CD39+ and CD39− subsets using magnetic activated cellular sorting (MACS). Cells were plated with indicated concentrations of platelet rich plasma and cultured for 24 hours in DMEM media. Supernatant was extracted and added to cultured HUVEC cells at 5% v/v concentration.

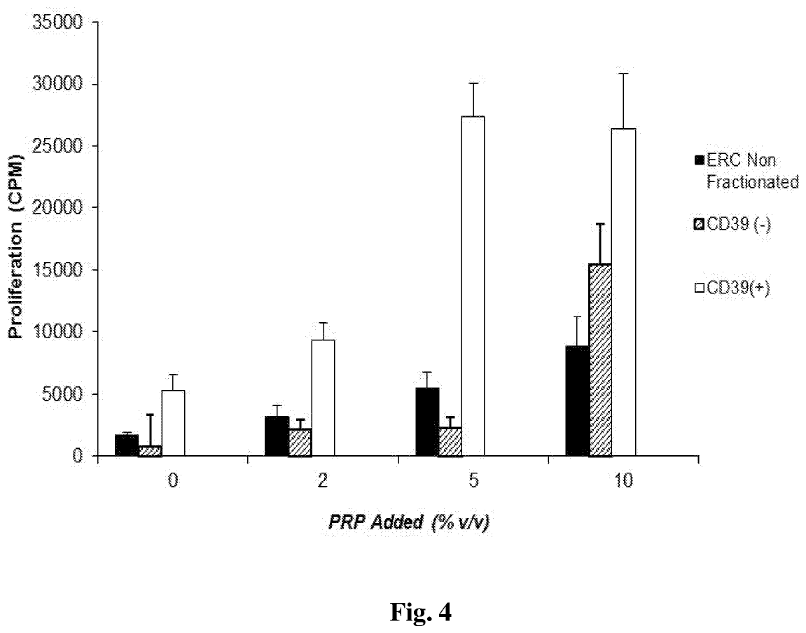

In order to replicate angiogenic processes in vitro, a culture of human umbilical vein endothelial cells (HUVEC) was performed in 96 well plates. HUVEC cells were plated at a concentration of 20,000 cells per well. Cells were subsequently incubated at the indicated ratio with HUVEC cells in a total of 200 uL of media per well for 72 hours. 1 uCurie/ml of tritiated thymidine was added in the last 12 hours of culture and proliferation is quantified by scintillation counting. Proliferation was expressed as counts per minute. The proliferation of non-fractionated ERCs, CD39+ ERCs and CD39− ERCs with and without treatment of PRP is shown in FIG. 4.

### REFERENCES

1. Milkiewicz, M., C. W. Pugh, and S. Egginton, Inhibition of endogenous HIF inactivation induces angiogenesis in ischaemic skeletal muscles of mice. J Physiol, 2004. 560(Pt 1): p. 21-6.
2. van Weel, V., et al., Expression of vascular endothelial growth factor, stromal cell-derived factor-1, and CXCR4 in human limb muscle with acute and chronic ischemia. Arterioscler Thromb Vasc Biol, 2007. 27(6): p. 1426-32.
3. Tuomisto, T. T., et al., HIF-VEGF-VEGFR-2, TNF-alpha and IGF pathways are upregulated in critical human skeletal muscle ischemia as studied with DNA array. Atherosclerosis, 2004. 174(1): p. 111-20.
4. Wang, Y., et al., Changes in circulating mesenchymal stem cells, stem cell homing factor, and vascular growth factors in patients with acute ST elevation myocardial infarction treated with primary percutaneous coronary intervention. Heart, 2006. 92(6): p. 768-74.
5. Scheufler, K. M., et al., Implications of vascular endothelial growth factor, sFlt-1, and sTie-2 in plasma, serum and cerebrospinal fluid during cerebral ischemia in man. J Cereb Blood Flow Metab, 2003. 23(1): p. 99-110.
6. Takeshita, S., et al., Therapeutic angiogenesis. A single intraarterial bolus of vascular endothelial growth factor augments revascularization in a rabbit ischemic hind limb model. J Clin Invest, 1994. 93(2): p. 662-70.
7. Takeshita, S., et al., Intramuscular administration of vascular endothelial growth factor induces dose-dependent collateral artery augmentation in a rabbit model of chronic limb ischemia. Circulation, 1994. 90(5 Pt 2): p. 11228-34.
8. Hopkins, S. P., et al., Controlled delivery of vascular endothelial growth factor promotes neovascularization and maintains limb function in a rabbit model of ischemia. J Vasc Surg, 1998. 27(5): p. 886-94; discussion 895.
9. Pu, L. Q., et al., Enhanced revascularization of the ischemic limb by angiogenic therapy. Circulation, 1993. 88(1): p. 208-15.
10. Sun, Q., et al., Sustained vascular endothelial growth factor delivery enhances angiogenesis and perfusion in ischemic hind limb. Pharm Res, 2005. 22(7): p. 1110-6.
11. van Weel, V., et al., Vascular endothelial growth factor overexpression in ischemic skeletal muscle enhances myoglobin expression in vivo. Circ Res, 2004. 95(1): p. 58-66.
12. Helisch, A. and W. Schaper, Angiogenesis and arteriogenesis--not yet for prescription. Z Kardiol, 2000. 89(3): p. 239-44.
13. Rajagopalan, S., et al., Regional angiogenesis with vascular endothelial growth factor in peripheral arterial disease: a phase II randomized, double-blind, controlled study of adenoviral delivery of vascular endothelial growth factor 121 in patients with disabling intermittent claudication. Circulation, 2003. 108(16): p. 1933-8.
14. McDonnell, K., et al., Vascular leakage in chick embryos after expression of a secreted binding protein for fibroblast growth factors. Lab Invest, 2005. 85(6): p. 747-55.
15. Gerwins, P., E. Skoldenberg, and L. Claesson-Welsh, Function of fibroblast growth factors and vascular endothelial growth factors and their receptors in angiogenesis. Crit Rev Oncol Hematol, 2000. 34(3): p. 185-94.
16. Bernotat-Danielowski, S., et al., Generation and localisation of monoclonal antibodies against fibroblast growth factors in ischaemic collateralised porcine myocardium. Cardiovasc Res, 1993. 27(7): p. 1220-8.
17. Iwakura, A., et al., Myocardial ischemia enhances the expression of acidic fibroblast growth factor in human pericardial fluid. Heart Vessels, 2000. 15(3): p. 112-6.
18. Ortega, S., et al., Neuronal defects and delayed wound healing in mice lacking fibroblast growth factor 2. Proc Natl Acad Sci USA, 1998. 95(10): p. 5672-7.
19. Comerota, A. J., et al., Naked plasmid DNA encoding fibroblast growth factor type 1 for the treatment of end-stage unreconstructible lower extremity ischemia: preliminary results of a phase I trial. J Vasc Surg, 2002. 35(5): p. 930-6.
20. Cao, R., et al., Angiogenic synergism, vascular stability and improvement of hind-limb ischemia by a combination of PDGF-BB and FGF-2. Nat Med, 2003. 9(5): p. 604-13.
21. Carmeliet, P. and R. K. Jain, Angiogenesis in cancer and other diseases. Nature, 2000. 407(6801): p. 249-57.
22. Pajusola, K., et al., Stabilized HIF-1alpha is superior to VEGF for angiogenesis in skeletal muscle via adeno-associated virus gene transfer. Faseb J, 2005. 19(10): p. 1365-7.
23. Rajagopalan, S., et al., Use of a Constitutively Active Hypoxia-Inducible Factor-1{alpha} Transgene as a Therapeutic Strategy in No-Option Critical Limb Ischemia Patients. Phase I Dose-Escalation Experience. Circulation, 2007.
24. Calvani, M., et al., Hypoxic induction of an HIF-1alpha-dependent bFGF autocrine loop drives angiogenesis in human endothelial cells. Blood, 2006. 107(7): p. 2705-12.
25. Eppley, B. L., J. E. Woodell, and J. Higgins, Platelet quantification and growth factor analysis from platelet-rich plasma: implications for wound healing. Plast Reconstr Surg, 2004. 114(6): p. 1502-8.
26. Jenis, L. G., R. J. Banco, and B. Kwon, A prospective study of Autologous Growth Factors (AGF) in lumbar interbody fusion. Spine J, 2006. 6(1): p. 14-20.
27. Kevy, S. V. and M. S. Jacobson, Comparison of methods for point of care preparation of autologous platelet gel. J Extra Corpor Technol, 2004. 36(1): p. 28-35.
28. Wang, Z., et al., Thrombopoietin regulates differentiation of rhesus monkey embryonic stem cells to hematopoietic cells. Ann N Y Acad Sci, 2005. 1044: p. 29-40.
29. Xie, X., et al., Thrombopoietin promotes mixed lineage and megakaryocytic colony forming cell growth but inhibits primitive and definitive erythropoiesis in cells isolated from early murine yolk sacs. Blood, 2003. 101(4): p. 1329-35.
30. Feugier, P., et al., Ex vivo expansion of stem and progenitor cells in co-culture of mobilized peripheral blood CD34+ cells on human endothelium transfected with adenovectors expressing thrombopoietin, c-kit ligand, and Flt-3 ligand. J Hematother Stem Cell Res, 2002. 11(1): p. 127-38.
31. Kawada, H., et al., Rapid ex vivo expansion of human umbilical cord hematopoietic progenitors using a novel culture system. Exp Hematol, 1999. 27(5): p. 904-15.
32. Won, J. H., et al., Thrombopoietin is synergistic with other cytokines for expansion of cord blood progenitor cells. J Hematother Stem Cell Res, 2000. 9(4): p. 465-73.
33. Wang, J., et al., In vitro hematopoietic differentiation of human embryonic stem cells induced by co-culture with human bone marrow stromal cells and low dose cytokines. Cell Biol Int, 2005. 29(8): p. 654-61.
34. Levac, K., F. Karanu, and M. Bhatia, Identification of growth factor conditions that reduce ex vivo cord blood progenitor expansion but do not alter human repopulating cell function in vivo. Haematologica, 2005. 90(2): p. 166-72.
35. Peschle, C., et al., Stringently purified human hematopoietic progenitors/stem cells: analysis of cellular/molecular mechanisms underlying early hematopoiesis. Stem Cells, 1993. 11(5): p. 356-70.
36. Maurer, A. M., et al., Ex vivo expansion of megakaryocytic cells. Int J Hematol, 2000. 71(3): p. 203-10.
37. Willems, R., et al., Establishment of serum free pre-colony forming unit assays for differentiation of primitive hematopoietic progenitors: serum induces early macrophage differentiation and inhibits early erythroid differentiation of CD34++CD38− cells. Ann Hematol, 2001. 80(1): p. 17-25.
38. Scheding, S., et al., Effective ex vivo generation of granulopoietic postprogenitor cells from mobilized peripheral blood CD34(+) cells. Exp Hematol, 2000. 28(4): p. 460-70.
39. Ivanovic, Z., Interleukin-3 and ex vivo maintenance of hematopoietic stem cells: facts and controversies. Eur Cytokine Netw, 2004. 15(1): p. 6-13.
40. Inderbitzin, D., et al., Interleukin-3 induces hepatocyte-specific metabolic activity in bone marrow-derived liver stem cells. J Gastrointest Surg, 2005. 9(1): p. 69-74.
41. Bohmer, R. M., IL-3-dependent early erythropoiesis is stimulated by autocrine transforming growth factor beta. Stem Cells, 2004. 22(2): p. 216-24.
42. Quesenberry, P., et al., Long-term marrow cultures: human and murine systems. J Cell Biochem, 1991. 45(3): p. 273-8.
43. Zhang, P. L., et al., Increased myelinating capacity of embryonic stem cell derived oligodendrocyte precursors after treatment by interleukin-6/soluble interleukin-6 receptor fusion protein. Mol Cell Neurosci, 2005.
44. Taga, T. and S. Fukuda, Role of IL-6 in the neural stem cell differentiation. Clin Rev Allergy Immunol, 2005. 28(3): p. 249-56.
45. Nakamura, M., et al., Role of IL-6 in spinal cord injury in a mouse model. Clin Rev Allergy Immunol, 2005. 28(3): p. 197-204.
46. Ficara, F., et al., IL-3 or IL-7 increases ex vivo gene transfer efficiency in ADA-SCID BM CD34+ cells while maintaining in vivo lymphoid potential. Mol Ther, 2004. 10(6): p. 1096-108.
47. Krawczenko, A., C. Kieda, and D. Dus, The biological role and potential therapeutic application of interleukin 7. Arch Immunol Ther Exp (Warsz), 2005. 53(6): p. 518-25.
48. Andre-Schmutz, I., et al., IL-7 effect on immunological reconstitution after HSCT depends on MHC incompatibility. Br J Haematol, 2004. 126(6): p. 844-51.
49. De Waele, M., et al., Growth factor receptor profile of CD34 cells in normal bone marrow, cord blood and mobilized peripheral blood. Eur J Haematol, 2004. 72(3): p. 193-202.
50. Lu, L. L., et al., [Effect of Tpo and/or IL-11 gene modified stromal cells on the expansion of CD34+ CD38− hematopoietic primitive progenitor cells]. Zhonghua Xue Ye Xue Za Zhi, 2003. 24(11): p. 589-92.
51. Momose, K., et al., Effects of interleukin-11 on the hematopoietic action of granulocyte colony-stimulating factor. Arzneimittelforschung, 2002. 52(11): p. 857-61.
52. Van der Meeren, A., et al., Administration of recombinant human IL11 after supralethal radiation exposure promotes survival in mice: interactive effect with thrombopoietin. Radiat Res, 2002. 157(6): p. 642-9.
53. U, K., et al., Preclinical ex vivo expansion of G-CSF-mobilized peripheral blood stem cells: effects of serum-free media, cytokine combinations and chemotherapy. Eur J Haematol, 2005. 74(2): p. 128-35.
54. McGuckin, C. P., et al., Thrombopoietin, flt3-ligand and c-kit-ligand modulate HOX gene expression in expanding cord blood CD133 cells. Cell Prolif, 2004. 37(4): p. 295-306.
55. Lu, S. J., et al., CD34+CD38− hematopoietic precursors derived from human embryonic stem cells exhibit an embryonic gene expression pattern. Blood, 2004. 103(11): p. 4134-41.
56. Streeter, P. R., L. Z. Dudley, and W. H. Fleming, Activation of the G-CSF and Flt-3 receptors protects hematopoietic stem cells from lethal irradiation. Exp Hematol, 2003. 31(11): p. 1119-25.
57. Aliotta, J. M., et al., Bone marrow production of lung cells: The impact of G-CSF, cardiotoxin, graded doses of irradiation, and subpopulation phenotype. Exp Hematol, 2006. 34(2): p. 230-41.
58. Jung, K. H., et al., Granulocyte colony-stimulating factor stimulates neurogenesis via vascular endothelial growth factor with STAT activation. Brain Res, 2006.
59. Kogler, G., et al., Cytokine production and hematopoiesis supporting activity of cord blood-derived unrestricted somatic stem cells. Exp Hematol, 2005. 33(5): p. 573-83.
60. Gangenahalli, G. U., et al., Stem cell fate specification: role of master regulatory switch transcription factor PU.1 in differential hematopoiesis. Stem Cells Dev, 2005. 14(2): p. 140-52.
61. Otani, T., et al., Erythroblasts derived in vitro from embryonic stem cells in the presence of erythropoietin do not express the TER-119 antigen. Exp Hematol, 2004. 32(7): p. 607-13.
62. Yao, M., et al., Ex vivo expansion of CD34-positive peripheral blood progenitor cells from patients with non-Hodgkin's lymphoma: no evidence of concomitant expansion of contaminating bcl2/JH-positive lymphoma cells. Bone Marrow Transplant, 2000. 26(5): p. 497-503.
63. Mobest, D., R. Mertelsmann, and R. Henschler, Serum-free ex vivo expansion of CD34(+) hematopoietic progenitor cells. Biotechnol Bioeng, 1998. 60(3): p. 341-7.
64. Crcareva, A., et al., Hematopoietic stem cells expanded by fibroblast growth factor-1 are excellent targets for retrovirus-mediated gene delivery. Exp Hematol, 2005. 33(12): p. 1459-69.
65. de Haan, G., et al., In vitro generation of long-term repopulating hematopoietic stem cells by fibroblast growth factor-1. Dev Cell, 2003. 4(2): p. 241-51.
66. Ratajczak, M. Z., et al., Effect of basic (FGF-2) and acidic (FGF-1) fibroblast growth factors on early haemopoietic cell development. Br J Haematol, 1996. 93(4): p. 772-82.
67. Kang, H. B., et al., Basic fibroblast growth factor activates ERK and induces c-fos in human embryonic stem cell line MizhES1. Stem Cells Dev, 2005. 14(4): p. 395-401.
68. Schwartz, R. E., et al., Defined conditions for development of functional hepatic cells from human embryonic stem cells. Stem Cells Dev, 2005. 14(6): p. 643-55.
69. Quito, F. L., et al., Effects of fibroblast growth factor-4 (k-FGF) on long-term cultures of human bone marrow cells. Blood, 1996. 87(4): p. 1282-91.
70. Grothe, C., et al., Fibroblast growth factor-20 promotes the differentiation of Nurr1-overexpressing neural stem cells into tyrosine hydroxylase-positive neurons. Neurobiol Dis, 2004. 17(2): p. 163-70.
71. McDevitt, T. C., M. A. Laflamme, and C. E. Murry, Proliferation of cardiomyocytes derived from human embryonic stem cells is mediated via the IGF/PI 3-kinase/Akt signaling pathway. J Mol Cell Cardiol, 2005. 39(6): p. 865-73.
72. Musaro, A., Growth factor enhancement of muscle regeneration: a central role of IGF-1. Arch Ital Biol, 2005. 143(3-4): p. 243-8.
73. Zumkeller, W. and S. Burdach, The insulin-like growth factor system in normal and malignant hematopoietic cells. Blood, 1999. 94(11): p. 3653-7.
74. Okajima, Y., et al., Insulin-like growth factor-I augments erythropoietin-induced proliferation through enhanced tyrosine phosphorylation of STATS. J Biol Chem, 1998. 273(36): p. 22877-83.
75. Miyazaki, M., et al., Propagation of adult rat bone marrow-derived hepatocyte-like cells by serial passages in vitro. Cell Transplant, 2004. 13(4): p. 385-91.
76. von Ruden, T. and E. F. Wagner, Expression of functional human EGF receptor on murine bone marrow cells. Embo J, 1988. 7(9): p. 2749-56.
77. Bracci-Laudiero, L., et al., CD34-positive cells in human umbilical cord blood express nerve growth factor and its specific receptor TrkA. J Neuroimmunol, 2003. 136(1-2): p. 130-9.
78. Simone, M. D., et al., Nerve growth factor: a survey of activity on immune and hematopoietic cells. Hematol Oncol, 1999. 17(1): p. 1-10.
79. Guo, Y., B. Graham-Evans, and H. E. Broxmeyer, Murine Embryonic Stem Cells Secrete Cytokines/Growth Modulators that Enhance Cell Survival/Anti-Apoptosis and Stimulate Colony Formation of Murine Hematopoietic Progenitor Cells. Stem Cells, 2005.
80. Chodorowska, G., A. Glowacka, and M. Tomczyk, Leukemia inhibitory factor (LIF) and its biological activity. Ann Univ Mariae Curie Sklodowska [Med], 2004. 59(2): p. 189-93.
81. Su, R. J., et al., Platelet-derived growth factor enhances expansion of umbilical cord blood CD34+ cells in contact with hematopoietic stroma. Stem Cells Dev, 2005. 14(2): p. 223-30.
82. Lucarelli, E., et al., Platelet-derived growth factors enhance proliferation of human stromal stem cells. Biomaterials, 2003. 24(18): p. 3095-100.
83. Yang, M., C. N. Chesterman, and B. H. Chong, Recombinant PDGF enhances megakaryocytopoiesis in vitro. Br J Haematol, 1995. 91(2): p. 285-9.
84. Ploemacher, R. E., et al., Bone morphogenetic protein 9 is a potent synergistic factor for murine hemopoietic progenitor cell generation and colony formation in serum-free cultures. Leukemia, 1999. 13(3): p. 428-37.
85. Zhang, J. and L. Li, BMP signaling and stem cell regulation. Dev Biol, 2005. 284(1): p. 1-11.
86. Jay, K. E., et al., Identification of a novel population of human cord blood cells with hema-topoietic and chondrocytic potential. Cell Res, 2004. 14(4): p. 268-82.
87. Chadwick, K., et al., Cytokines and BMP-4 promote hematopoietic differentiation of human embryonic stem cells. Blood, 2003. 102(3): p. 906-15.
88. Dormady, S. P., et al., Immortalized multipotential mesenchymal cells and the hematopoietic microenvironment. J Hematother Stem Cell Res, 2001. 10(1): p. 125-40.
89. Shav-Tal, Y. and D. Zipori, The role of activin a in regulation of hemopoiesis. Stem Cells, 2002. 20(6): p. 493-500.
90. Cerdan, C., A. Rouleau, and M. Bhatia, VEGF-A165 augments erythropoietic development from human embryonic stem cells. Blood, 2004. 103(7): p. 2504-12.
91. Laharrague, P., et al., High expression of leptin by human bone marrow adipocytes in primary culture. Faseb J, 1998. 12(9): p. 747-52.
92. Gaspar Elsas, M. I., et al., Murine myeloid progenitor responses to GM-CSF and eosinophil precursor responses to IL-5 represent distinct targets for downmodulation by prostaglandin E(2). Br J Pharmacol, 2000. 130(6): p. 1362-8.
93. Grafte-Faure, S., et al., Effects of glucocorticoids and mineralocorticoids on proliferation and maturation of human peripheral blood stem cells. Am J Hematol, 1999. 62(2): p. 65-73.

What is claimed is:

1. A cellular population derived from the endometrium, containing fibroblastoid-like morphology, selected for enhanced expression of CD39 as compared to other cellular populations derived from endometrium containing fibroblast-like morphology.

2. The cellular population of claim 1, wherein said cell possesses adherence to plastic.

3. The cellular population of claim 1, wherein said cell population possesses ability to stimulate angiogenesis.

4. The cellular population of claim 1, wherein said CD39 expressing cell population possesses ability to secrete VEGF at an enhanced amount compared to other cell populations derived from the endometrium that are negative for expression of CD39.

5. The cellular population of claim 1, wherein said wherein said CD39 expressing cell population possesses ability to secrete HGF at an enhanced amount compared to other cell populations derived from the endometrium that are negative for expression of CD39.

6. The cellular population of claim 1, wherein said CD39 expressing cell population possesses ability to secrete PDGF-BB at an enhanced amount compared to other cell populations derived from the endometrium that are negative for expression of CD39.

7. The cellular population of claim 1, wherein endometrial derived cells are treated with platelet rich plasma to enhance expression of CD39.

8. The cellular population of claim 7, wherein said platelet rich plasma is a composition comprising of: a) platelets derived from whole blood at a first concentration of at least about 1.1 times a platelet concentration in the whole blood; b) white blood cells derived from the whole blood at a second concentration of at least a white blood cell concentration in the whole blood, wherein the white blood cells comprise: neutrophils at a third concentration, wherein the third concentration is less than the neutrophil concentration in the whole blood; c) lymphocytes at a fourth concentration of at least 1.1 times a lymphocyte concentration in the whole blood; and d) monocytes at a fifth concentration of about 1.1 times a monocyte concentration in the whole blood.

9. The cellular population of claim 8, wherein the third concentration is 1% of the concentration of neutrophils in whole blood.

10. The cellular population of claim 8, wherein the neutrophils are substantially eliminated.

11. The cellular population of claim 8, wherein the third concentration is between about 2,000 neutrophils per microliter and about 3,000 neutrophils per microliter.

12. The cellular population of claim 8, wherein monocytes and/or lymphocytes are increased in comparison to neutrophils in said platelet rich plasma composition.

13. The cellular population of claim 12, wherein neutrophils are depleted to less than 3000 neutrophils per microliter.

14. The cellular population of claim 8, wherein said platelet rich plasma is buffered to a physiological pH, and wherein the physiological pH is between about 7.3 and about 7.5.

15. The cellular population of claim 8, wherein said platelet rich plasma contains cytokines.

16. The cellular population of claim 15, wherein said cytokines are selected from a group comprising of: a) IL-1; b) IL-6; and c) TNF-alpha.

17. The cellular population of claim 8, wherein said platelet rich plasma contains chemokines.

18. The cellular population of claim 17, wherein said chemokines are selected from a group comprising of: a) ENA-78; b) MCP-3; c) IL-8; d) MIP-1 alpha; e) NAP-2; f) CXCL4; g) CCL5; and h) SDF-1.

19. The cellular population of claim 8, wherein said platelet rich plasma contains growth factors capable of stimulating angiogenesis.

20. The cellular population of claim 19, wherein said growth factors capable of stimulating angiogenesis are selected from a group comprising of: a) angiopoietin; b) FGF-1; c) FGF-2; d) HGF; e) PDGF-BB; and f) VEGF.

21. The cellular population of claim 1, wherein said cells are cultured in a media selected from a group comprising of: a) Roswell Park Memorial Institute (RPMI-1640); b) Dublecco's Modified Essential Media (DMEM), c) Eagle's Modified Essential Media (EMEM), d) Optimem, and e) Iscove's Media.

22. The cellular population of claim 1, wherein said cells are endowed with additional angiogenic potency by culture with agents selected from a group comprising of: a) low dose IL-2; b) IL-10; c) TGF-beta; d) IL-35; e) VEGF; f) PDGF; g) CTLA-4 Ig; and h) anti-CD45RB antibody.

23. The cellular population of claim 1, wherein said cell is endowed with additional immune modulatory activity, by culture with agents selected from a group comprising of: a) IL-10; b) PGE-2; c) aspirin; d) a statin; e) n-acetylcystein; f) rapamycin; g) IVIG; and h) naltrexone.
